(19) United States
(12) Patent Application Publication
(10) Pub. No.: US 2003/0003554 A1
(43) Pub. Date: Jan. 2, 2003

(54) Novel cell culture supports with particular properties, and production thereof

(76) Inventors: Alain Othon Andre Miller, Mons (BE); Nathalie Liliane Paule Ghislaine Ropson-Kenda, Emines (BE)

Correspondence Address: YOUNG & THOMPSON, 745 SOUTH 23RD STREET 2ND FLOOR, ARLINGTON, VA, 22202

(21) Appl. No.: 10/133,325

(22) Filed: Apr. 29, 2002

Related U.S. Application Data
(63) Continuation of application No. PCT/EP00/11245, filed on Oct. 27, 2000.

(30) Foreign Application Priority Data
Oct. 29, 1999 (EP) 99402710.0

(51) Int. Cl. C12N 11/00; C12N 11/16

(52) U.S. Cl. 435/174

(57) ABSTRACT

A method for continuously obtaining two-dimensional microcarrier beads (2D-MS) displaying particular functional characteristics for anchorage-dependent cell culture (CAD), includes a succession of steps which consist in: continuously producing polymer film rolls from a specific polymer granules; activating the polymer film by generating reactive groups; covalent grafting between the activated polymer film and a polymer, copolymer or a macromolecule of interest; if required, washing to eliminate the monomers which have not been consumed and fixed on the film, followed by drying the grafted film; and finally cutting the grafted polymer film by continuous punching, the size of the punch being selected according to that of the dimension desired for the two-dimensional microcarrier beads.

The present invention relates to a novel process for producing two-dimensional (2D) microsupports for culture of anchorage-dependent cells. More precisely, the invention relates to a process for preparing such microsupports suitable for mass culture, by perfusion or in clusters of animal anchorage-dependent cells and which manifest particular properties such as cryosensitivity, biocompatibility, biodegradability or specific adhesion. The invention also relates to microsupports obtained by the process and to their use. Finally, it relates to a device for the mass production of these microsupports of homogeneous size and constitution.

Anchorage-dependent cells (ADC) are dependent on adhesion to a support for their proliferation and for retaining their cellular functions and viability. This dependence constitutes a technological stumbling block for the production of biological and pharmaceutical substances secreted by ADCs over those from anchorage-independent cells, which can proliferate in suspension. This dependence originates primarily from the fact that the growth of ADCs stops when confluence occurs and the confluent cells have to be detached by trypsinisation. Further, the cells' need for nutrient medium and oxygen limits the number and/or surface area of the microsupports to a given volume of culture medium. Different culture systems have been developed with a view to providing a sufficient anchorage surface area and to allow ADCs to be produced on an industrial scale: roller bottles, multi-tray systems, hollow fibres and microsupports, which are particularly advantageous as regards the surface area/volume ratio.

A number of microsupports used in the industry for the mass cultivation of anchorage-dependent cells (ADC) are characterized by a three-dimensional geometry (3D); the easiest to produce have a spherical geometry. 3D microbeads are used by the biopharmaceutical industry to batch produce cell cultures (bioreactors up to 1000 l); an example is Cytodex® sold by Pharmacia (UPPSALA, Sweden) suspended in an amount of 5 g/l to obtain concentrations of the order of 2×10⁶ cells/ml, 7% by volume of culture being occupied by the microbeads. They constitute the industry standard. However, the surface area/volume ratio of spherical microsupports is not conducive to increasing the concentration of the microsupports in the bioreactor. If high concentrations of ADC are to be cultivated (10⁷ to 5×10⁷ cells/ml), the concentration of microsupport in the bioreactor must be capable of being increased. However, a point is quickly reached at which the volume of the swollen microbeads represents too high a proportion of the culture volume, reducing the volume of medium available to the cells.

A new generation of microsupports with a two-dimensional geometry has been developed and described in European patent EP-A-0 579 596.

The term “two-dimensional geometry” means that the thickness of these microsupports tends to become infinitesimal and negligible compared with the dimensions of the cultivated cells. This reduction in thickness is such that there is no possibility of cell growth either in the support or on its edge, but only on the two anchorage faces. Such 2D microsupports (2D-MS) offer the principal advantage of an anchorage surface per unit volume that is higher than all 3D competitors such as the CYTODEX® type microbeads mentioned above.

Thus, for a given culture volume occupation in a bioreactor, they can cultivate and produce more cells per unit volume. The external anchorage surface area of a sphere (4nR²) is equal to the total surface area of two infinitely thin disks (2×2nR²) located at the equator and inscribed in that sphere. However, the combined volumes of these two “equatorial” disks reduces as the thickness of the film used to produce them reduces, representing only an infinitesimal fraction of the volume of the sphere that circumscribes them. By adopting a thickness of 10 μm for all of the disks generated in a sphere, the total surface area suitable for anchorage of the cells thus provided by the disks is about 3.3 times higher than the external surface area of the sphere, taking their random motions in suspension into account.

The large anchorage surface area available for ADCs per unit volume of 2D-MS thus allows high concentration cell culture on an industrial scale to be envisaged.

A further limitation on supports for anchorage-dependent cell culture is that of the method used to recover the cells attached to their support while retaining the biological or physiological properties of the cells. The cells are detached from their support using an enzymatic treatment (for example trypsin) or a chelating agent (for example EDTA), which can damage not only the cell functions but also their subsequent re-attachment to supports when continuous culture is performed. This limitation is a particular problem when the biological functions of the cells are then used on an industrial scale. This situation is frequently encountered when producing macromolecules of interest using those cells, or when the integrality of the receptors or membrane molecules is desired for their capacity to bind ligands or internalise molecules or substances.

Further, culture to confluence of anchorage-dependent cells leads to the formation of intercellular bonds or intercellular junctions. These intercellular junctions also contribute to the properties of the cells and their destruction contribute to the loss of these properties.

These limitations on the mass culture of anchorage-dependent cells also result in a limit in the industrial production of biological macromolecules produced by those cells, whether they are molecules normally synthesised by the cells in question or more generally produced by insertion of genes coding for a heterologous protein using genetic recombination techniques. This limitation connected to the production capacities of functional anchorage-dependent cells can result in cost prices for proteins to be expressed and purified that are incompatible with the subsequent sale price of a drug containing that protein as an active principle.

Thus, there is a need for the provision of microsupports for ADC culture with the following specific features:

high yield of cells per unit volume of culture medium;

minimum alteration to the viability of ADCs following detachment from the microsupports;

control of the number and specificity of cells that can anchor to said supports, that control enabling the supports to be adapted to the culture systems used and to the properties of those cultivated cells;

possibility of large scale production of those microsupports for anchorage-dependent cells with excellent reproducibility and at a cost price that is compatible with the sale price for the production of cells or macromolecules of biological interest.

Attempts have been made to overcome a certain number of the disadvantages described above, in particular the need to use enzymes or chelating agents to detach the cells from their support, namely in EP 0 387 975 and EP 0 382 214. These two patents propose coating conventional cell culture supports with the product of copolymerisation of hydrophilic monomers selected from poly-N-alkyl(meth)acrylamide derivatives, their respective copolymers, poly-N-acryloyl-piperidine or poly-N-acryloyl-pyrrolidine, the desired property of which is cryosensitivity.

In that use, the polymer is selected as a function of the lower critical temperature or LCST, which is a transition temperature for hydration and dehydration of the polymer compound. When the LCST is lower than the cell culture temperature, the cells remain fixed on the polymer support during the cell culture phase. They can be detached by reducing the temperature of the culture so that it is substantially lower than the LCST. Methods for producing a polymer or copolymer with a given low LCST have been described in EP-B1-0 382 214. All the polymers grafted from the monomers cited in that patent application are suitable but are not restrictive in the application to cryosensitivity in the present application. In general, it can be said that including hydrophilic monomers in the polymerisation process tends to increase the LCST, while the presence of hydrophhobic monomers tends to reduce the LCST. Examples of hydrophilic monomers are: N-vinyl pyrrolidone, vinylpyridine, acrylamide, methacrylamide, N-methyl-acrylamide, hydroxyethyl-methacrylate, hydroxyethyl acrylate, hydroxymethyl-methacrylate, hydroxymethyl-acrylate, acrylic acid and methacrylic acid containing acid groups and their salts, vinylsulphonic acid, styrylsulphonic acid and N,N′dimethylamino-ethyl-methacrylate, N,N′-diethylamino-ethyl-methacrylate and N,N′-dimethylamino-propyl-acrylamide containing basic groups, and salts thereof.

Examples of hydrophobic monomers are: acrylate and methacrylate derivatives such as ethyl acrylate, methyl methacrylate and glycidyl methacrylate, etc., N-substituted-alkyl (meth)acrylamides derivatives such as N-n-butyl(meth)acrylamide and N-isopropyl acrylamide, etc., as well as vinyl chloride, acrylonitrile, styrene and vinyl acetate, etc.

However, the means described for coupling such cryosensitive polymers or copolymers to the cell culture supports cannot:

either control the quantity and thus the thickness of the grafted polymers, a particularly important parameter when the density of the cell culture has to be controlled;

or ensure covalent coupling of the polymer or copolymer to the culture support; this is a major disadvantage as it means that the supports cannot be stored for long periods, nor can they be re-used.

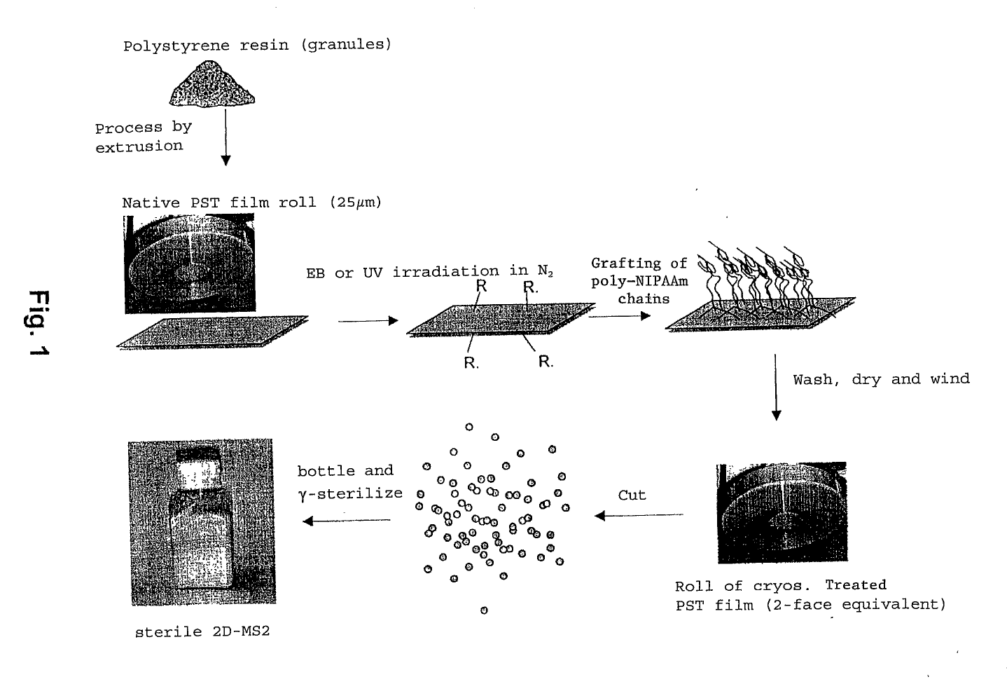

The present invention provides a process for continuously producing two-dimensional (2D) supports endowed with particular properties for anchorage-dependent cell culture (ADC). It is illustrated in FIG. 1 and comprises at least the following steps in succession:

continuously producing rolls of polymer film with a thickness of 35 μm or less from granules of a given polymer;

activating said polymer film using any means for generating reactive groups, in particular radicals or peroxide functions, or hydroperoxide functions, or amine functions;

producing covalent grafts between the activated polymer film and a polymer, a copolymer or a macromolecule of interest the property of which is desired, said grafting being achieved by immersing the film in a solution of monomer, copolymerisation being initiated by free radicals created by activation under β irradiation, the immersion time being directly correlated to the desired thickness of the polymer grafted onto the film;

if necessary, washing to eliminate monomers that are not consumed and not fixed to the film;

cutting the polymer film by a process selected as a function of the desired geometry and size of the 2D supports;

if necessary, a sterilisation step, either by autoclaving or by irradiation, the sterilisation method clearly being selected as a function of the material to be sterilised. When the material cannot be autoclaved, it must be sterilised using a physical method (γ or β irradiation) or a chemical method (isopropanol/H₂O, 70/30%, v/v).

The polymer film can be cut by any suitable means depending on the nature of the film. Polystyrene films, for example, have a number of aromatic groups and are suitable for photoablation using an excimer laser.

Despite the relatively poor quality of this type of cutting, it is possible to envisage cutting by pyrolysis (argon laser), for example using cellophane disks coloured red using a non toxic substance.

A particularly advantageous process comprises cutting by continuous punching, the size of the punches being that of the desired 2D microsupports.

The combination of these different steps, including activation followed by covalent grafting of a processed polymer film (substrate) then cutting the substrate film carrying the polymer, copolymer or macromolecule covalently grafted by a process for producing 2D microsupports that are homogeneous in size and geometry, constitutes the original nature of the invention.

Regarding the starting material employed in the form of a film, the polymer can be of any nature. In accordance with the invention, it is desirable for the material to:

have a density in the range 0.9 to 1.25 g/cm³, preferably in the range 1 to 1.1 g/cm³ to allow agitated culture in suspension in a culture medium (in a bioreactor) with no risk of sedimentation or flotation;

be transparent to allow ready analysis of the cells during their growth, continuously in the bioreactor. The term “transparent” means that for wavelengths in the range 400 nm to 1000 nm, light traverses the microsupports with no substantial attenuation of the intensity of the emergent light beam compared with the intensity of the incident light beam. An adsorption of less than 1% is considered to be entirely advantageous;

be characterized by a suitable hydrophobic/lipophilic balance; if it is more hydrophobic, it must have a contact angle so that is it sufficiently wettable in an aqueous medium and if it is rather hydrophilic, it must not swell in water; in other words, the contact angle θ between the surface of the material and a droplet of aqueous medium must be in the range 30° to 90°, the contact angle being defined as the angle formed between the surface of the material and the tangent to the droplet at the triple intersection point between the droplet, the surface of the material and the air.

An angle θ=0 corresponds to perfect wetting and the liquid surface is parallel to the material surface. An angle θ of >90° corresponds to an absence of wetting and the droplets remained formed on the material surface. A contact angle θ in the range 30° to 90° corresponds to imperfect wetting, corresponding to partial spreading of the droplet on the material.

In accordance with the invention, the polymer material that can be used to produce the films (substrate) can be polystyrene, polyethylene, polyethyleneterephthalate or polycarbonate, or any copolymer mainly including these rather hydrophobic polymers. A more hydrophilic film can be cellophane or an aliphatic polyester such as polylactide or polyhydroxybutyrate and any copolymer mainly including these more hydrophilic materials.

When the polymer employed is an aliphatic polyester type, the film is in essence bioresorbable/biodegradable and in this case, it can be used as an implant into a living organism if the polymer employed is of biomedical grade, preferably recognised by the FDA.

Preferably, the film used to produce the microsupports is between 10 and 25 μ thick.

Thin or ultra-thin polymer rolls are continuously produced using the “extrusion-drawing” technique starting from granules of a given polymer. This starting material is heated to melt it, then extruded through a rotating screw and moulded between two plates to produce a thick polymer film. At the outlet from the extruder, the fairly thick film is then hot drawn either in a single direction or in two orthogonal directions to produce a non-shrinkable film with a much reduced thickness that can be controlled (10 to 35 μm) over the entire width of the produced roll, within the limits of the thermal and mechanical properties of the starting material.

The “extrusion-blowing” technique is an alternative for producing the film continuously. The variation lies in the second step, namely injection of air between two walls of films which stretches the material and thus produces a reduced film thickness. Other techniques for producing thin films have been described in other applications of polymer chemistry (such as biosensors): spin coating or solvent casting are two, but these techniques are usually employed to produce small disks or sheets and are less suited to the production of continuous rolls of film.

In a second step, the polymer film, which can advantageously be in a windable form with a width in the range 5 cm to 60 cm and a length of at least 3 km, then undergoes activation to enable reactive groups to be generated that can form covalent bonds with other reactive groups of the substance that is to be grafted. In this context, the term “substance” means organic monomers or polymers with particular properties, in particular cryosensitivity or biocompatibility; they may also be biological macromolecules which have a specific affinity for certain cell receptors: the 2D microsupports resulting from such grafting can then allow selective adhesion of certain types of cells present in an initial cell sample comprising a mixture of cells. By way of example, skin cells (keratinocytes) can be cited, at different stages of differentiation, or cells resulting from insertion, activation or repression of a particular function, in particular by insertion of a gene carrying said function or carrying a function regulating expression of a cellular gene.

Four processes are employed to modify the chemical structure of the polymers and generate reactive groups. They are electron beams, more particularly β irradiation, corona discharges, UV treatment and, finally, plasmas. For each procedure, two parameters govern the choice of method depending on the desired properties for the material undergoing the irradiation:

the nature of the chemical groups induced in the polymer by activation;

the depth of treatment into the thickness of the material.

In all cases, activation consists of subjecting the support to electromagnetic irradiation that causes bonds to break and free radicals to be created, either peroxide functions, hydroperoxide functions or amine functions.

Using the methods described, spacer molecules can if required be grafted via the free radicals generated. They function to increase the length of the bond between the reactive sites and the monomers, polymers or macromolecules to be covalently bonded to the polymer film, and as a result increase their mobility.

Activation consists of subjecting the film to electron bombardment. Bombardment is preferably accomplished in an inert atmosphere. In the process of the invention, it is essential that the activation step precedes the grafting step. When these two steps are simultaneous, as is the case in EP 382 214, the monomer to be grafted is then subjected to irradiation to create a very large number of free homopolymers in solution which can adsorb onto the surface of the support and which must then be eliminated by washing. In this case, it is then difficult to ensure that all of the free, non-grafted chains are eliminated by washing, and that the adsorbed free chains do not dissolve in the culture medium during cellular detachment by thermal contrast.

The activation conditions are selected as a function of a certain number of parameters including at least:

the nature of the polymer film to be grafted;

the nature of the copolymer or macromolecules to be covalently coupled to the polymer film;

the discontinuous or continuous nature of the activation process; the grafted support that will then be cut may have been treated in a static manner or continuously by passage of the film.

When the activation process is continuous, the film passage rate can be from 0.1 to 50 m per minute and can be established as a function of the total dose of irradiation required to activate the film and of the power of the irradiator, fixed as a function of the thermal resistance of the film.

When irradiating polystyrene film with β or γ rays, the irradiator power can be increased to 6 milliamps (mA); beyond that, the film overheats and deforms. If the rate of film passage is substantially reduced, then for a fixed intensity of 6 mA, it is possible to achieve maximum doses of 80 to 200 kGrays in a single passage beneath the irradiator.

The kiloGray or joule per kilogram is a unit representing the dose and depends on the characteristics of the electron beam unit.

When a corona discharge is used, it is emitted at a tension of several thousand volts at frequencies in the kHz region. This process is carried out in an ambient atmosphere. The geometric amplitude of the corona arc is from a few millimeters for the more conventional systems to a few centimeters for blown arc systems. Discharge is achieved using parallel electrodes located at either side of the article. The use of corona discharges to activate the film has the advantage of being a treatment carried out in an ambient atmosphere.

The polymer film can also be activated by pre-irradiation with UV. This procedure is carried out in the presence of a photo-initiator. As an example, the film can be exposed to a high pressure mercury lamp for one hour in the presence of acetone gas carrying benzophenone acting as the photo-initiator at 40° C. in nitrogen.

Activating the plasma film by cold plasma is also carried out using electrodes that emit discharges in the radiofrequency region.

A plasma is obtained by ionisation using a high frequency source of a gas or a mixture of gas introduced into a chamber under a residual pressure of a few millibars. This expensive procedure is also difficult to carry out continuously on a polymer film. However, these four types of activation: β or γ electron beam, corona discharge, UV or plasma are suitable for generating reactive groups.

In accordance with the invention, the technique of radiografting acrylamide or vinyl type monomers as described above to the surface of a thin aliphatic polystyrene or polyester type polymer film must be initiated by irradiation regardless of the nature thereof. The grafting step is then carried out directly by immersion by plunging the pre-activated polymer film into a solution of monomer, a mixture of a plurality of monomers or of selected macromolecules.

If an organic monomer is used, its copolymerisation to the surface of the polymer film is initiated by the free radicals created during pre-irradiation, and the polymer chains formed are bonded covalently to the film. The reaction is instantaneous but, depending on the nature of the film, it can be prolonged to a few seconds or a few minutes to produce a maximum degree of grafting. This degree of grafting is directly linked to the irradiation dose, to the concentration of monomers in the impregnating bath, and to the reaction time.

The process of the invention then comprises, inter alia, optimising the four parameters cited above, namely: nature and dose of irradiation, grafting period, concentration of monomers and nature of solvent in the grafting bath, to obtain a layer of covalently grafted polymer, copolymer or specific macromolecules of the desired thickness. As will be shown in Example 2 below, the thickness of the deposited polymer layer, organic copolymer layer or specific macromolecule layer is determined by X ray photon spectroscopy (XPS) and depends directly on the duration of the grafting step by impregnation in the bath.

When the pre-irradiation step is carried out in air, the reactive groups created are susceptible of being instantaneously oxidised in the presence of the air. In this case, the grafting step is carried out extemporaneously by immersion in a bath containing an ad hoc compound to regenerate the reactive radicals.

In the process of the invention, the grafting step can if necessary be followed by a washing step consisting of eliminating monomer, polymer or biological macromolecule residues that have not been consumed and/or polymerised to the surface of the polymer film. These washes are generally carried out in a mixture of isopropanol in water (70/30% v/v) until there is no further trace of reactants and/or products in the washings. When applied to cell culture, it is essential that washing should be as efficient as possible because of the cytotoxicity of acrylamide monomers and polymers and derivative thereof.

In the process of the invention, different natures of polymers, copolymers or macromolecules can constitute a layer of 1 to 10 nanometers to which adherent cells fix and proliferate. When total or partial cryosensitivity is sought, the polymer or copolymer of interest is selected from derivatives of poly-N-alkyl(meth)acrylamides, their respective copolymers, poly-N-acryloyl piperidine and poly-N-acryloyl pyrrolidine. When biocompatibility is desired, the surface treatment involves, for example, covalent grafting of a hydrophilic polymer such as an amine-containing polyethylene oxide PEO to the surface of a polymer film pre-exposed to an allylamine plasma, to generate amine functions on the surface, via a suitable chemical coupling agent such as cyanide chloride. This type of treatment has been described in J. Biomed. Mater. Res. 1991, 25, 1547.

When the desired property is the selective adhesion of animal cells, conventional methods for grafting macromolecules onto supports such as those described in affinity chromatography techniques using antibodies, aptamers or molecules obtained by combinatory chemistry can be used. The skilled person can find a detailed description of these techniques in J. Cell. Biol. 1991, 114, 1089 §1990, 110, 777, J. Biol. Chem. 1992, 267, 14019 §10133, Artif. Organs 1992, 16, 526, Macromolecules, 1993, 26, 1483. Biological macromolecules (oligopeptides, oligonucleotides, etc) that can advantageously be grafted onto the supports prepared by the process of the invention are specific ligands for cell receptors that enable to perform the growing of a certain type of cell in a culture medium to the detriment of other cell types that could be mixed with them. More particularly, it can allow multiplication of cells that express a specific macromolecule in their membrane, either naturally or as a result of in vitro genetic recombination.

In the process of the invention, the substrate polymer film on which a polymer, copolymer or macromolecule of interest is grafted is then cut using a process selected as a function of the desired geometry and size of the 2D microsupports, and as a function of the nature of the polymer film.

A preferred implementation of the present invention is punching, the size of the punches matching that of the 2D microsupports produced.

In one preferred implementation of the invention, the microsupport thickness is preferably 25 μm or less and it is in the form of a disk. The last step in the process for producing such grafted 2D-MS thus comprises cutting the thin or ultra-thin grafted polymer film (substrate) into micrometric particles characterized by a two-dimensional geometry and comprising two anchorage faces on which the cells attach and proliferate without any penetration of the cells between the two faces.

One preferred cutting technique is cutting by mechanical punching, which produces an excellent quality of microdisks as regards homogeneity of size, shape and thickness of the microdisks produced, and in terms of an absence of debris.

Homogeneity of size (homodispersity) is essential to synchronising the different steps in cell growth. The presence of different sizes of microsupports would mean that the smaller ones would reach confluence before the larger ones. In this case, the ADCs that attained confluence earlier could partially detach, die and release ammonia, lactic acid and other toxins that are deleterious to the growth of ADCs proliferating on larger microsupports.

This step is accomplished by punching the uniquely pre-activated or pre-activated then grafted polymer film with circular ceramic carbide punches of a selected diameter (for example 150 μm). The polymer film is punched at a cutting frequency (impacts per minute) that is optimised as a function of the rate of advancement of the polymer film under consideration.

Other cutting techniques can be envisaged, such as laser photoablation, pyrolysis cutting, or embossing systems using a rotary knife constituted by fixed lines on a printing cylinder of a given diameter, a second cylinder of the same diameter acting as a press. In this latter technique, a given number of lines of punches are distributed over the 360° of the cylinder (defined as a function of the spacing between punches and the cylinder diameter), also a given number of punches per line (defined as a function of the length of the cylinder). The cutting rate for a roll of film passing between the two cylinders and thus the productivity must be different from the present process. This is a powerful technique used by companies selling all sizes and shapes of labels. In this case, the pressure that has to be applied to produce a cut risks limiting the process.

The invention also concerns two-dimensional (2D) microsupports endowed with particular properties for mass culture of anchorage-dependent cells (ADC) obtained by a process as described above, characterized in that the thickness of the polymer film is in the range 10 to 35 μm, and the thickness of the covalently grafted polymer, copolymer or macromolecule of interest is in the range 1 to 10 nm.

The succession of steps in the production of the grafted 2D-MS is shown in FIG. 1.

When the 2D-MS are cryosensitive, a minimum thickness of 5 nanometers for the grafted layer is necessary if cells at confluence are to detach themselves quantitatively from their support. Reduced thicknesses are required when wishing partial and non quantitative detachment of the ADCs at confluence, by thermal contrast.

The invention also concerns a device for continuous preparation of a 2D-MS support endowed with particular properties, prepared using the process defined above and employing covalent fixing of a polymer, a copolymer or of biological macromolecules on a substrate constituted by a polymer film, said device comprising:

a system for unwinding/winding a polymer film, the film being entrained at a selected speed for each step: activation, grafting, punching;

an irradiator to activate the film surface;

a grafting pond for containing the solution of monomers, polymers or macromolecules to be grafted, into which the pre-activated polymer film continuously passes at a predetermined speed entrained by the winding/unwinding system;

a cutting tool for punching the film as it is unwound.

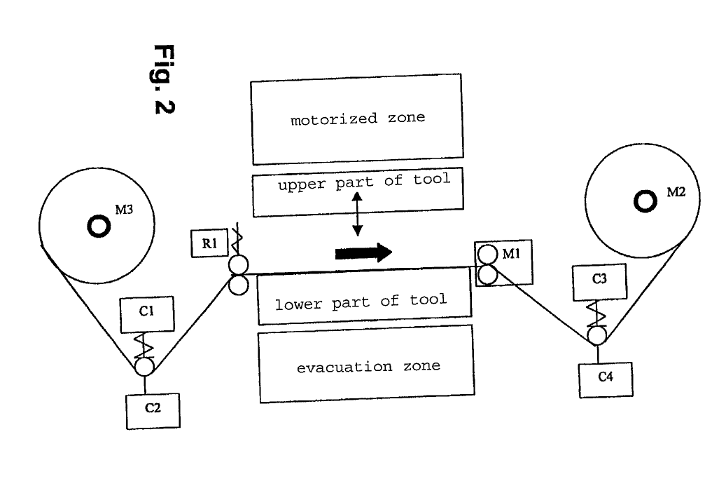

FIG. 2 shows a diagram of an device for continuous cutting of a film (in the form of rolls). The winding/unwinding system serves to supply to and evacuate from a cutting tool. Further, this tool will operate 24 hours a day. M1 is the motor governing strip advance; M2 is the motor for winding the roll and M3 is the motor for unwinding the roll. C1 represents the sensor for operating M3, C2 is the stop sensor for M3, C3 represents the sensor for operating M2 and C4 is the stop sensor for M2. Finally, R1 indicates the strip braking system.

Preferably, the film unwinding/winding system can advance said film at a rate in the range 5 to 50 m per minute. In some cases, the film can be advanced more slowly, and the rate of this advance is in the range 0.05 to 8 m per minute. The unwinding/winding rate is much slower in the cutting step than during the activation and grafting steps.

The core of the device is constituted by a tool block on which are locked, in the film length direction, rows of circular punches and a corresponding recess block. By way of example, for a film that is 5 cm in width, the tool block will comprise 9 rows each with 50 punches. This in-line tool block configuration in the longitudinal direction rather than the width direction increases maintenance safety. In the same line of punches, the spacing is 0.25 mm; the regular distance between 2 lines is 0.20 mm. Clearly, the above disposition, the punch diameter and their spacing is an optimum proposition but can, of course, be adapted to requirements and is not limiting in nature. The general geometrical disposition of the punches can also be optimised.

As was seen above, the cutting device can be constituted by a laser.

During punching, the grafted 2D-MS microsupports are continuously recovered. As an example, from a grafted polystyrene roll with a width of 5 cm and a length of 3 km, the device of the invention comprising punches with a diameter of 150 μm can produce about 1 kg of disks (2D-MS microsupports) 150 μm in diameter. Knowing that the surface density of the material is 26.25 g/m² the minimum number of particles obtained is 2.38×10⁹ microsupports per roll or per kilo of microsupports produced. In the configuration cited above as an example, the cutting yield is 28%.

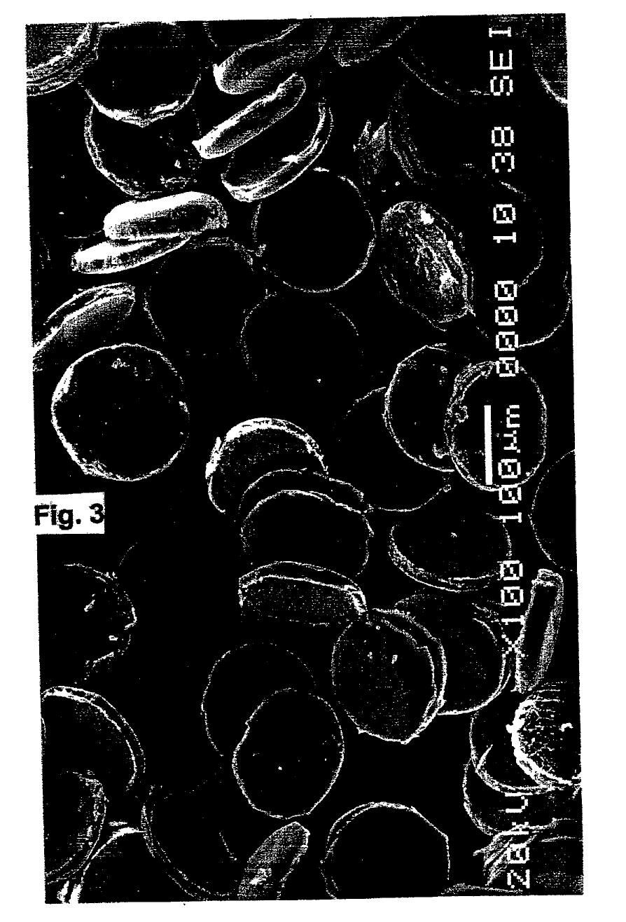

FIG. 3 shows the 2D-MS microsupports obtained.

The microdisks can be recovered in receptacles positioned directly beneath the punching matrix.

The non-limiting examples below show the advantages of the process and the supports for cell culture as obtained by the process.

### EXAMPLE 1

### Radiografting Via Pre-Irradiation Using an Electron Beam in Nitrogen

Firstly, we sought to optimise and define the discontinuous radio-grafting parameters (total activation dose, nature of solvent, concentration of monomer, temperature of grafting bath, grafting period) on sheets (film of 5×10 cm², 25 μ thick polystyrene) with a research static electron accelerator of the Van der Graaf type characterized by a low power and low dose rate compared with industrial device. These sheets were initially placed in pre-degassed 75 cm³ culture flasks and dry irradiated in an inert atmosphere (nitrogen) as follows:

The culture flasks containing the polystyrene film were washed twice in a 70/30% (v/v) isopropanol/H₂O mixture and dried for 15 minutes in a stream of nitrogen.

The degassed polystyrene culture flasks were placed in a static high energy EB irradiator (10 meV). A van der Graaf electron accelerator (10 meV) with a fixed intensity of 1 mA and a dose rate of 10 kGray/min (1 Mrad/min), was used. The flasks were then irradiated under the beam for a fixed time (in minutes) to absorb a set total dose (in kGray or Joule/g). The dose deposited was previously calibrated using a dosimeter.

At the outlet from the irradiator, the flasks were immediately brought into contact with the solution (H₂O), monomer stock (concentration 10% to 40% by weight) in a nitrogen atmosphere. The stock solution of monomer, freshly prepared, was transferred to the pre-irradiated polystyrene flask by a pressurised nitrogen system. Once transferred into the pre-irradiated flask, the grafting solution was equilibrated at a given temperature (25° C. to 60° C.). The grafting time (0.5 to 24 hours) was varied at a 20 given temperature to verify the influence of various parameters on the thickness of the layer of poly-N-isopropylacrylamide on the surface of the film as on the polystyrene culture flask. The grafting solutions were eliminated from the flasks after a given time then the flasks and the grafted films were washed three times and dried.

### EXAMPLE 2

### Grafting of N-Isopropylacrylamide (NIPAAm) to a Polystyrene Film

2.1 Study of Deposit Thickness as a Function of Reaction Time

25 micron thick films were irradiated in nitrogen using a Van der Graaf type electron accelerator (10 meV) and a total dose of 250 kGray. The irradiated films were then immediately immersed in an aqueous NIPAAm solution (10% by weight) which had been degassed for 30 min with nitrogen. The temperature of the grafting solution was 60° C. Different grafted surfaces were obtained by varying the concentration of monomer in the grafting solution and the reaction time. When the grafting reactions were complete, the grafted films were washed, dried and analysed by X ray photon spectroscopy (XPS) to determine the chemical composition of the external surface.

The deposit thickness as a function of reaction time is shown in Table 1 below.

TABLE 1
Films grafted in Example 1 - XPS analysis

| | | | | | % cellular |
|---|---|---|---|---|---|
| | | | | Deposit | detach- |
| Film samples | Experimental atomic % | | | thickness | ment |
| (grafting time) | C | O | N | (Å) | (counting) |
| Native polystyrene | 98.5% | 1.5% | 0% | — | — |
| Grafted polystyrene | 92.3% | 4.1% | 3.6% | 38 Å | 65.5% |
| (½ h) | | | | | |
| Grafted polystyrene | 78.7% | 11.3% | 10% | 47 Å | 79.0% |
| (1 h) | | | | | |
| Grafted polystyrene | 75.7% | 13.1% | 11.2% | >50-60 Å | 96.5% |
| (3 h) | | | | | |
| Native poly-N- | 75.0% | 12.7% | 12.3% | — | — |
| isopropylacrylamide | | | | | |

The C₁ₛ peak of native polystyrene can be resolved into two components (at 284.8 eV and 291.5 eV) which respectively correspond to the carbon involved in the hydrocarbon bonds and to the “shake-up” peak characteristic of aromatic compounds. According to the literature, this latter peak has an intensity of 7% with respect to the total carbon for polystyrene.

The film thickness was calculated from the ratio of the surface area (%) of the characteristic shake up peak Cis of polystyrene and of the total C₁ₛ peak for the grafted film sample analysed at an electron collection angle of 900° (quantitative analysis). In the case where the characteristic C₁ₛ shake up peak of pure polystyrene was no longer detected in XPS, the thickness of the grafted deposit was greater than the analysis depth of the XPS technique, namely 50 or 60 Å.

The inventors also demonstrated the importance of a further activation parameter, the irradiation temperature, to obtain grafting with a suitable deposit thickness (5 to 10 nm). The lower the set temperature at which the irradiation chamber was kept by controlled injection of liquid nitrogen, the faster the grafting kinetics (the larger the quantity of active sites retained on the surface) and the shorter the grafting time could be to obtain a suitable deposit thickness (in this example, the time was reduced from 3 h to 2 h when the irradiation temperature was changed from 0° C. to −20° C.).

Further, the purity of the commercial monomer dissolved in water (10% by weight) was also critical to obtain effective grafting. The presence of traces (0.1% by weight) of a stabilising agent, methylhydroquinone (MHQ) in the solid monomer produced a yellow coloration in the prepared aqueous solution and above all impeded copolymerisation of the NIPAAm to the surface of the polystyrene film: no cryosensitive deposit was observed after grafting in this non-purified solution. In order to obtain an optimum cryosensitive deposit during the continuous process, the inventors developed a process for industrial scale decolorisation of the freshly dissolved aqueous solution of monomer by selective adsorption of the contaminant on an activated charcoal column (purification carried out just prior to the film pre-activation step). On the laboratory scale, the alternative to clarification/decolorisation of the aqueous 10% by weight monomer solution is re-crystallisation of the commercially available solid NIPAAm from a toluene/heptane mixture. In this case, the stabilising agent, which remained soluble in the organic phases, was eliminated after several washes of the re-crystallised monomer. However, for the continuous process (where batches of several tens of liters of solutions are used, and thus several kg of NIPAAm), the activated charcoal purification technique is more appropriate.

The skilled person will use any available technique that is estimated to be the most suitable for eliminating MHQ, and these techniques should be considered to be functional equivalents to the techniques described above.

2.2 25 μm thick films were discontinuously irradiated in nitrogen using a Van der Graaf type electron accelerator (10 meV) with a total dose of 150 kGray. The irradiated films were immediately immersed in a solution of NIPAAm in isopropanol (10% by weight) which had been degassed for 30 minutes with nitrogen, for 3 hours. The temperature of the grafting solution was 20° C. No deposition of polyN-IPAAm was observed.

2.3 25 μm thick films were discontinuously irradiated in air using an Electrocurtain type electron accelerator operating at 150 keV with a total dose of 100 kGray. The irradiated films were immediately trapped in liquid nitrogen (during transport) and stored at very low temperature (freezer at −180° C.) to preserve the stability of the peroxide/hydroperoxide functions generated on the surface. After defrosting, the pre-irradiated films were immediately immersed in an aqueous NIPAAm solution (10% by weight) containing ferrous chloride (0.1% by weight) which had been degassed for 30 minutes with nitrogen. This reducing agent chemically decomposed the oxidised functions on the film surface to sites initiating radical polymerisation of NIPAAm. The temperature of the grafting solution was 37° C. The monomer/reducing agent mole ratio and the reaction time had an effect on the grafting yield and the deposit thickness.

2.4 25 μm thick films one of the two faces of which had been masked with an aluminium sheet were irradiated in nitrogen using a Van der Graaf type electron accelerator (10 meV) and with a total dose of 250 kGray. The irradiated films were immediately immersed in an aqueous NIPMm solution (10% by weight) which had been degassed for 30 minutes with nitrogen. The temperature of the grafting solution was 60° C. Supports selectively grafted on only one of the two faces were obtained.

We can conclude from these different tests that the highest grafting yields leading to a deposit thickness of more than 5 nanometers were obtained during a pre-irradiation step with a total irradiation dose of 250 kGray (irradiation time 25 min) by leaving the pre-activated films in contact with the monomer for at least 2 hours. When the grafting time was shorter, the grafting yields and as a result the thickness of the deposit obtained on the polystyrene film were smaller (less than 5 nanometers). Such surfaces only partially provided the desired properties and functions.

### EXAMPLE 3

### Radiografting Using Pre-Irradiation with UV in Nitrogen

Polystyrene films were pre-irradiated in UV using a high pressure mercury lamp for 1 h in the presence of an acetone gas carrying benzophenone acting as a photo-initiator, at 40° C., in nitrogen. The irradiated films were then immediately immersed in an aqueous NIPAAm solution (10% by weight) which had been degassed with nitrogen for 30 min. The temperature of the grafting solution was 60° C. and the grafting time was 3 h.

Using this technique, it was possible to graft poly-N-isopropylacrylamide to the surface of a polystyrene film.

1. A process for continuously producing two-dimensional (2D) supports endowed with particular properties for anchorage-dependent cell culture (ADC), comprising at least the following steps in succession:
continuously producing rolls of polymer film with a thickness of 35 μ or less and with a density in the range 0.9 to 1.25 g/cm³ from granules of a given polymer;
activating said polymer film using any means for generating reactive groups, in particular radicals and/or peroxide, hydroperoxide or amine functions;
producing covalent grafts between the activated polymer film and a polymer, a copolymer or a macromolecule of interest the property of which is desired, said grafting being achieved by immersing the film in a solution of monomer, copolymerisation being initiated by free radicals created by activation under β irradiation, the immersion time being directly correlated to the desired thickness of the polymer grafted onto the film;
if necessary, washing to eliminate monomers that are not consumed and not fixed to the film, followed by drying the grafted film;
cutting the grafted polymer film by continuous punching, the size of the punches being selected as a function of the desired size of the 2D supports.

2. A process according to claim 1, in which the contact angle θ of the polymer film is in the range 30° to 90°.

3. A process according to claim 1, in which activation is accomplished by corona discharge, plasma, UV irradiation or electron bombardment.

4. A process according to claim 3, in which the polymer film is electronically activated by bombardment with β irradiation in an inert atmosphere.

5. A process according to claims 1 to 4, in which the thickness of the polymer layer grafted onto the film surface is determined by a combination of the following parameters: the total dose of p irradiation received by the film, the concentration of monomer in the grafting tank, the grafting time and the grafting temperature.

6. A process according to any one of the preceding claims, in which the polymer or copolymer of interest is selected from derivatives of poly-N-alkyl(meth)acrylamides, poly-N-isopropylacrylamide (NIPAAm), their respective copolymers, poly-N-acryloyl piperidine and poly-N-acryloyl pyrrolidine the desired property of which is cryosensitivity.

7. A process according to claims 1 to 5, in which the polymer or copolymer of interest is a hydrophilic amine-containing polyethylene oxide PEO type polymer the desired property of which is biocompatibility.

8. A process according to claim 6, in which all traces of stabilising agent are eliminated from the monomer solution.

9. A process according to claims 1 to 5, in which the macromolecule(s) of interest is (are) one or more specific ligands for cell receptors, the desired property of which is the selective adhesion of cells carrying this (these) receptor(s).

10. A process according to claim 6 in which, when the polymer film (substrate) is polystyrene and when the grafted polymer is selected from derivatives of poly-N-alkyl(meth)acrylamides, their respective copolymers, poly-N-acryloyl piperidine or poly-N-acryloyl pyrrolidine, a thickness of 40 to 60 Å is obtained by a combination of a total dose of irradiation of 50 to 250 Kgrays, and an immersion time and temperature in the monomer solution of 1 to 3 hours and 50 to 70 degrees respectively.

11. A process according to claim 1, in which activation is prior to grafting of the polymer or copolymer of interest with the desired property.

12. Two-dimensional (2D) microsupports endowed with particular properties for mass culture of anchorage-dependent cells (ADC) obtained by a process according to any one of the preceding claims, characterized in that the thickness of the polymer film is in the range 10 to 35 microns, and the thickness of the covalently grafted polymer, copolymer or macromolecule of interest is in the range 1 to 10 nm.

13. Microsupports according to claim 12, in which the grafted polymer is a cryosensitive polymer selected from derivatives of poly-N-alkyl(meth)acrylamides, their respective copolymers, poly-N-acryloyl piperidine and poly-N-acryloyl pyrrolidine.

14. Microsupports according to claim 12, in which the grafted polymer is a hydrophilic amine-containing polyethylene oxide PEO type polymer.

15. Microsupports according to claim 12, in which the grafted macromolecule(s) is (are) one or more specific ligands for cell receptors.

16. A device for continuous preparation of a 2D-MS support endowed with particular properties, prepared using the process defined above and employing covalent fixing of a polymer, a copolymer or of biological macromolecules on a substrate constituted by a polymer film, said device comprising:
a system for unwinding/winding a polymer film, the film being entrained at a selected speed for each step: activation, grafting, punching;
an electron accelerator arranged to continuously activate the rolls of film;
a receptacle for containing the solution of monomers, polymers or macromolecules to be grafted, into which the polymer film continuously passes at a predetermined speed entrained by the winding/unwinding system;
a cutting tool for punching the film as it is unwound.

17. A device according to claim 16, in which the film unwinding/winding system can advance said film at a speed in the range 0.05 to 8 m per minute.

18. A device according to claim 16, in which the film unwinding/winding system can advance said film at a speed in the range 5 to 50 m per minute.

19. A device according to any one of claims 16 to 18, in which the cutting tool is constituted by a tool block on which are locked, in the longitudinal direction of the film, rows of circular punches of ceramic carbide, and a corresponding recess block.

20. A device according to any one of claims 16 to 18, in which the cutting tool is a laser.

21. A device according to any one of claims 16 to 18, in which the cutting tool is constituted by a rotary cylindrical knife (embossing system).
